(19) United States
(12) Patent Application Publication
(10) Pub. No.: US 2009/0317529 A1
(43) Pub. Date: Dec. 24, 2009

(54) Aromachemicals and Processes for Preparation

(75) Inventor: Luca Turin, Somerville, MA (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Flexitral, Inc., Purcellville, VA (US)

(21) Appl. No.: 12/484,767

(22) Filed: Jun. 15, 2009

Related U.S. Application Data
(60) Provisional application No. 61/074,279, filed on Jun. 20, 2008.

(51) Int. Cl. A23L 1/226; C07C 255/23; C07C 255/31; A23L 2/56; C12G 3/06

(52) U.S. Cl. 426/534; 558/443; 558/434; 426/590; 426/592

(57) ABSTRACT

Aromachemical compounds of Formula I, compositions comprising these compounds and processes for their preparation are disclosed,

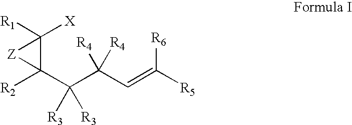

wherein R₁ is H, C(O)O—CH₂—CH₃; C(O)O—CH₃, or C(O)O—H; R₂ is methyl; R₃ and R₄ are H; R₅ and R₆ are CH₃; Z is C(H)₂; and X is —CN. The double bond attached to X can be in the E or Z configuration.

### FIELD OF THE INVENTION

The present invention relates generally to the field of flavors and fragrances. More particularly, the present invention relates to aromachemical compounds and processes for their preparation.

### BACKGROUND OF THE INVENTION

A variety of flavors and fragrances (i.e., aromachemicals) for use as ingredients in perfumes exist in the marketplace. Many aromachemicals, however, include double bonds or other potentially susceptible reactive groups that may result in a limited useful lifetime. Essential oil fragrances may exhibit undesirable properties which mean they are potentially harmful to human health resulting in allergic reactions, while some may be toxic, genotoxic, or carcinogenic. Aromachemicals that have novel or improved fragrance profiles and other physical properties are particularly useful for use as fragrances now exist such as those described in U.S. Publication No. 2007/0276152 and U.S. Publication No. 2007/0264340. These novel aromachemicals, however, present a unique challenge from a production standpoint. Cyclopropanation of an olefin usually requires the use of carbenes which must be generated by a variety of methods, e.g. halocarbenes, sulfoxonium compounds, Simmons-Smith reaction etc. Each of these methods suffer from high reagent costs and relatively low yields. The process described herein provides an efficient, safe and cost-effective process for preparing these aromachemicals.

### SUMMARY OF THE INVENTION

A process for preparing a compound of Formula I is provided,

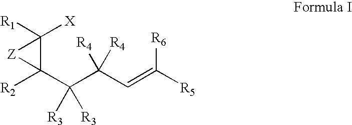

wherein R₁ is H, C(O)O—CH₂—CH₃; C(O)O—CH₃, or C(O)O—H;

R₂ is methyl;

R₃ and R₄ are H;

R₅ and R₆ are CH₃;

Z is C(H)₂; and

X is —CN. The double bond attached to X can be in an E or Z configuration.

The process comprises reacting a cyanoacetate with a ketone in a condensation reaction to produce the compound of Formula I′

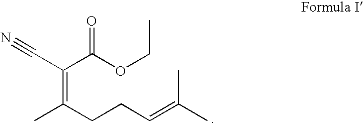

The cyanoacetate can be ethyl cyanoacetate. The ketone can be 6-methyl-5-hept-2-one.

The process further comprises the step of cyclopropanating the compound of Formula I′ to produce the compound of Formula II′:

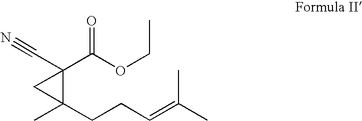

The process further comprises the step of reacting the ester of Formula II′ to produce the carboxylic acid of Formula III′.

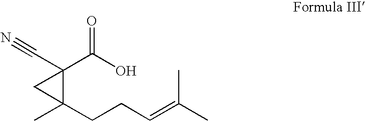

The step of reacting the ester of Formula II′ to produce the carboxylic acid of Formula III′ can be accomplished by hydrolysis. The hydrolysis can be catalyzed by at least one compound chosen from an acid or base or a combination thereof.

The process further comprises the step of decarboxylating the carboxylic acid of Formula III′ to form the compound of Formula IV′.

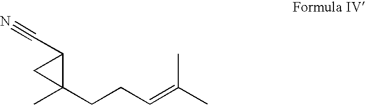

The step of decarboxylating the carboxylic acid can be accomplished in a variety of ways including: heating to a temperature of from 150° C. to 250° C.; heating to a temperature of from 110° C. to 150° C. in the presence of dimethylformamide; reacting the compound of Formula III′ with 1,3 propane diol in the presence of NaHCO₃ and heat in the range of 150° C. to 210° C.; or heating to a temperature of from 110° C. to 150° C. in the presence of toluene.

A composition is also provided comprising at least one compound of Formula I

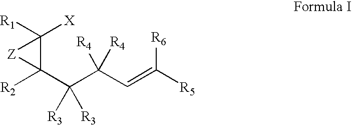

wherein R₁ is C(O)O—CH₂—CH₃; C(O)O—CH₃, or C(O)O—H;

R₂ is methyl;

R₃ and R₄ are H;

R₅ and R₆ are CH₃;

Z is C(H)₂; and

X is —CN.

The at least one compound of Formula I is in admixture with at least one solvent or adjuvant or a combination or mixture thereof. The composition can comprise at least one article chosen from a perfume, a cologne, a beverage, a soap, a bath or shower gel, a shampoo or other hair care product, a cosmetic preparation, a body deodorant, an antiperspirant, an air freshener, a fabric detergent or softener or an all-purpose household cleaner. The beverage can be a beer, malt liquor, lemonade or cola.

### DETAILED DESCRIPTION OF THE INVENTION

In one embodiment, the process for preparing compounds of Formula I comprises the step of preparing a compound of Formula I′.

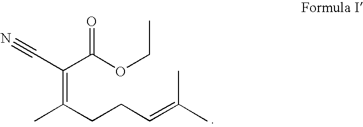

The compounds of Formula I′ can be produced by reacting a cyanoacetate with a ketone in a condensation reaction. The cyanoacetate may comprise a variety of suitable alkyl substituents. In one embodiment, the cyanoacetate is ethyl cyanoacetate, illustrated below as compound [A]. In one embodiment, the ketone is 6-methyl-5-hept-2-one, illustrated below as compound [B]. The reaction is illustrated as follows:

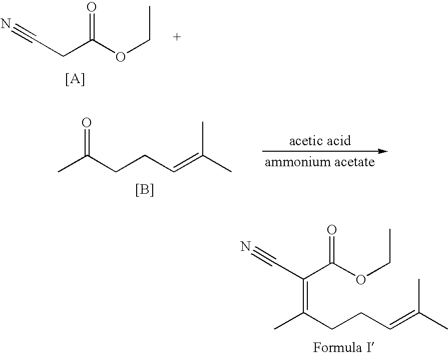

Conditions suitable for the condensation reaction, for example, include those found in the Gewald reaction which contains a Knoevenagel reaction step or those found in an aldol condensation of a ketone. In one embodiment, the condensation reaction is conducted in the presence of a buffer solution. The buffer solution may comprise any suitable acid and the salt of a weak acid or base. In one embodiment, the acid is acetic acid and the salt of a weak acid or base is ammonium acetate. The condensation reaction can be carried out in anhydrous toluene under reflux conditions.

In one embodiment, the process of preparing compounds of Formula I includes the step of cyclopropanating the compound of Formula I′ to produce the compound of Formula II′, illustrated as follows:

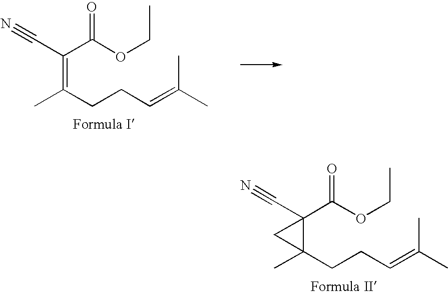

Suitable methods of cyclopropanation include, but are not limited to, carbenoid reactions such the Simmons-Smith cyclopropane synthesis (see for example Vogel's textbook of Practical Organic Chemistry 5^{th} Edition (1989) pp 1106-1108 or Solomon's Organic Chemistry 4^{th} Edition pp 346 and 347, published by John Wiley and Sons). The Simmons-Smith reactions proceeds by a single-step cycloaddition of a methylene (CH₂) unit from the reagent to the alkene. In the Simmons-Smith reaction, the reagent can be iodomethylzinc iodide which is prepared by the reaction of zinc-copper couple (ZnCu) with diiodomethane in ether.

In one embodiment, the monocyclopropanated compound of Formula II′ can be synthesized by subjecting the compound of Formula I′ to a haloform reaction to produce the dichloro or dibromo cyclopropyl derivative followed by dehalogenation with, e.g., lithium to provide the desired product.

In another embodiment, a Friedrichs reaction can be used to prepare the cyclopropanated product of Formula II′ (see, for example, Friedrich & Lewis, J. Org. Chem., 1990, 55, 2491-2494). In this reaction, acetyl chloride is used to accelerate the cyclopropanation of an alkene with a 1,1,-dibromo or 1,1-diiodo alkyl such as dibromomethane or diiodomethane using zinc dust and copper (I) in ether. The Friedrichs reaction is preferred for preparing the compounds of Formula II′ from starting compounds such as geraniol or nerol.

In another embodiment, the step of cyclopropanation is carried out by adding nitromethane and 1,8-Diazabicyclo[5.4.0]undec-7-ene to the compound of Formula I′ in the presence of anhydrous acetronitrile to produce the compound of Formula II′.

In one embodiment, the process of preparing compounds of Formula I includes the step of converting the ester of Formula II′ to the carboxylic acid of Formula III′, illustrated as follows:

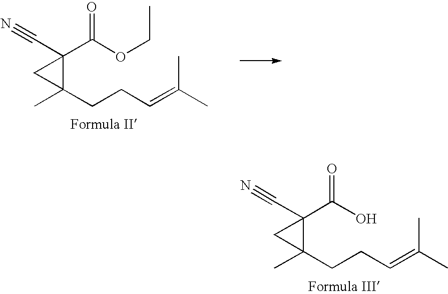

This step can include the hydrolysis or breakdown of the ester of Formula II′. The hydrolysis yields an alcohol and a carboxylic acid. In one embodiment, the carboxylic acid is the compound of Formula III′.

The hydrolysis can be catalyzed by at least one acid or at least one base or a combination thereof (i.e., saponification). Suitable bases include hydroxides, nitrogenous bases, oxides of Group I, Ca, Sr, or Ba, and conjugate bases of weak acids. Suitable nitrogenous bases include, but are not limited to, ammonia and hydroxylated nitrogenous bases such as, 2-aminobutanol, aminoethyl propanediol, aminomethyl propanol, aminopropanediol, bis-hydroxyethyl tromethamine, butyl diethanolamine, butylethanolamine, dibutyl dthanolamine, diethanolamine, diisopropanolamine, diisopropylamine, dimethyl isopropanolamine, monoethanolamine, dimethyl monoethanolamine, ethyl ethanolamine, isopropanolamine, isopropylamine, methyleth-anolamine, methylglucamine, morpholine, triethanolamine, triispropanolamine, tromethamine and combinations or mixtures thereof.

Other bases suitable for use include hydroxides such as calcium hydroxide, lithium hydroxide, potassium hydroxide, and sodium hydroxide; metal oxides such as calcium oxide, and sodium oxide; and conjugate bases of weak acids such as dipotassium phosphate, disodium phosphate, magnesium carbonate, pentapotassium triphosphate, petnasodium trisphosphate, potassium carbonate, sodium carbonate, tetrapotassium pyrophosphate, tetrasodium pyrophosphate, trisodium phosphate and combinations or mixtures thereof.

Suitable acids used to neutralize either an excess of nitrogenous base or a secondary nitrogenous base may generally be selected from the acid classes of anhydrides and organic and inorganic acids. Appropriate organic compounds include, but are not limited to, carboxylic acids, organic acid anhydrides and mixed acid anhydrides. Suitable neutralizing acids include linear carboxylic acids such as acetic acid, lactic acid, and glycolic acid; homocyclic carboxylic acids such as acetylsalicylic acid; hetrocyclic carboxylic acids such as nicotinic acid; aromatic carboxylic acids such as benzoic acid; branched aliphatic carboxylic acids such as isopropanoic acid; polyprotic carboxylic acids such as oxalic acid and succinic acid; and organic and mixed anhydrides such as benzoic acid anhydride and mixed phosphoanhydride. Suitable inorganic acids may include, but are not limited to, strong and weak polyprotic acids such as sulfuric acid and phosphoric acid; monoprotic weak acids such as sodium bisulfate; monoprotic strong acids such as hydrogen halides and perchloric acid; inorganic acid anhydrides such as carbon dioxide as well as combinations for mixtures thereof.

In one embodiment, the process of preparing the compounds of Formula I includes the step of decarboxylating the carboxylic acid of Formula III′ to form the compound of Formula IV′, illustrated as follows:

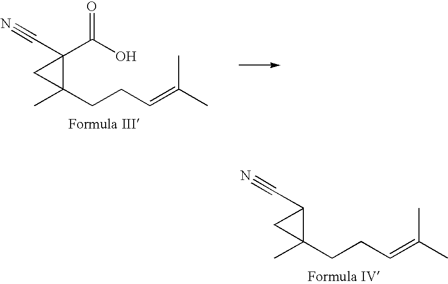

The carboxyl group of Formula III′ can be removed using a variety of suitable methods. In one embodiment, the carboxylic acid compound of Formula III′ is mixed with solid soda lime, and the resulting mixture is heated.

In another embodiment, the carboxyl group can be removed by reacting the carboxylic acid of Formula III′ with bromine in the presence of silver salts. The bromine replaces the carboxyl group leaving both the carboxyl carbon atom and the remaining organic moiety oxidized (Hunsdiecker reaction). Alternatively, lead tetraacetate is used to catalyze decarboxylation thus leaving the atoms of the organic residue in their original oxidation states.

In one embodiment, the carboxyl group of Formula III′ can be removed under neat conditions by heating the compound of Formula Ill′ to a temperature of from 100° C. to 300° C. for about 1 to 4 hours.

In another embodiment, the carboxyl group of Formula III′ can be removed under neat conditions by heating the compound of Formula III′ to a temperature of from 125° C. to 275° C. for about 1 to 4 hours.

In yet another embodiment, the carboxyl group of Formula III′ can be removed under neat conditions by heating the compound of Formula III′ to a temperature of from 150° C. to 250° C. for about 1 to 4 hours.

In one embodiment, the carboxyl group of Formula III′ can be removed in the presence of dimethylformamide by heating to a temperature of from 60° C. to 200° C. for about 1 to 4 hours.

In another embodiment, the carboxyl group of Formula III′ can be removed in the presence of dimethylformamide by heating to a temperature of from 85° C. to 175° C. for about 1 to 4 hours.

In yet another embodiment, the carboxyl group of Formula III′ can be removed in the presence of dimethylformamide by heating to a temperature of from 110° C. to 150° C. for about 1 to 4 hours.

In one embodiment, the carboxyl group of Formula III′ can be removed by reacting the compound of Formula III′ with 1,3 propane diol in the presence of NaHCO₃. In such an embodiment, the mixture can be heated to a temperature of from 100° C. to 260° C. for about 1 to 4 hours.

In another embodiment, the mixture can be heated to a temperature of from 125° C. to 235° C. for about 1 to 4 hours

In yet another embodiment, the mixture can be heated to a temperature of from 150° C. to 210° C. for about 1 to 4 hours

In one embodiment, the carboxyl group of Formula III′ can be removed by heating with toluene to a temperature of from 60° C. to 200° C. for about 1 to 4 hours.

In another embodiment, the carboxyl group of Formula III′ can be removed by heating with toluene to a temperature of from 85° C. to 175° C. for about 1 to 4 hours.

In yet another embodiment, the carboxyl group of Formula III′ can be removed by heating with toluene to a temperature of from 110° C. to 150° C. for about 1 to 4 hours.

The compounds of Formula I can be used to confer, improve, enhance or modify the taste or flavor property of a composition, product, preparation or article. A method to confer, improve, enhance or modify the aroma, fragrance or odor characteristics of compositions, products, preparations or articles comprises adding thereto an aroma, fragrance or odor effective amount of a composition or mixture of compounds of Formula I.

The compounds of Formula I can be included in virtually any article of manufacture that can include fragrance or flavorant compounds. Examples include hypochlorite (bleach) compositions, detergents, flavorings and fragrances, beverages, including alcoholic beverages. The compounds of Formula I can be used in personal care applications such as soaps, shampoos, denture cleanser tablets, body deodorants and antiperspirants. The compounds of Formula I can also be used as solid or liquid detergents for treating textiles, fabric softeners, detergent compositions and/or all-purpose cleaners for cleaning dishes or various surfaces, for both household and industrial use and candles. The use of the compounds is not limited to the above-mentioned products, as they may be used in other current uses in perfumery, namely the perfuming of soaps and shower gels, hygiene or hair-care products, as well as of body deodorants, air fresheners and cosmetic preparations, and even in fine perfumery, namely in perfumes and colognes.

The compounds of Formula I find utility in foods, flavorings, beverages such as beer and soda, denture cleansers (tablets), flavored orally-delivered products such as lozenges, candies, chewing gums, matrices, pharmaceuticals and the like.

The compounds of Formula I can be used as perfuming ingredients, as single compounds or as mixtures thereof. The compounds can be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all of the above applications, the compounds of Formula I can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through their general knowledge and as a function of the nature of the product to be perfumed and of the desired olfactive effect. These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin.

The proportions in which the compounds of Formula I can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the compounds of Formula I are typically present at concentrations between about 0.01 and about 30%, or even more, by weight of these compounds relative to the weight of the composition, product or article in which they are incorporated. It will be appreciated that the amount by weight of a compound of Formula I in a particular composition or product will depend on the nature of the composition. For example, a washing powder will typically contain less than 1% by weight of a compound of Formula I while a fine fragrance may contain more than 20% by weight of a compound of Formula I.

The compounds of Formula I may be used in detergents such as those containing bleaching agents and activators such as, for example, tetraacetylethylenediamine (TAED), hypohalites, in particular hypochlorite, peroxygenated bleaching agents such as, for example, perborates, etc. The compounds of Formula I can also be used in body deodorants and antiperspirants, for example, those containing aluminum salts.

The compositions described herein may include a detersive surfactant and optionally, one or more additional detergent ingredients, including materials for assisting or enhancing cleaning performance, treatment of the substrate to be cleaned, or to modify the aesthetics of the detergent composition (e.g. perfumes, colorants, dyes, etc.). Non-limiting examples of synthetic detersive surfactants useful herein typically at levels from about 0.5% to about 90%, by weight, include the conventional C₁₋₁₈ alkyl benzene sulfonates (“LAS”) and primary, branch-chain and random C₁₀₋₂₀ alkyl sulfates (“AS”), and the like. In one embodiment, compositions incorporating only synthetic detergents have a detergent level of from about 0.5% to 50%. In another embodiment, compositions containing soap preferably comprise from about 10% to about 90% soap. The compositions can contain other ingredients such as enzymes, bleaches, fabric softening agents, dye transfer inhibitors, suds suppressors, and chelating agents, all well known within the art.

The compounds of Formula I can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and/or fruit juice concentrates.

Artificial colorants that may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts that may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

The beverage may, for example, be a carbonated cola beverage. The pH is generally about 2.8 and the following ingredients can be used to make the syrup for these compositions: Flavor Concentrate, including one or more of the compounds of Formula I herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water.

Flavored food and pharmaceutical compositions including one or more of the compounds of Formula I can also be prepared. The compounds can be incorporated into conventional foodstuffs using techniques well known to those of skill in the art. Alternatively, the compounds can be incorporated within polymeric particles, which can, in turn, be dispersed within and/or over a surface of an orally-deliverable matrix material, which is usually a solid or semi-solid substrate. When used in chewable compositions, the compounds of Formula I can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

Flavored compositions may include an orally-deliverable matrix material; a plurality of water insoluble polymeric particles dispersed in the orally-deliverable matrix material, where the polymeric particles individually define networks of internal pores and are non-degradable in the digestive tract; and one or more compounds of Formula I entrapped within the internal pore networks. The compounds of Formula I are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a compound or a mixture of compounds prepared according to the process as described above to a desired flavor concentration. In one method for producing such gum based products a blade mixer is heated to about 110° F., the gum base is preheated so that it is softened, and the gum base is then added to the mixer and allowed to mix for approximately 30 seconds. The compound or compounds of Formula I are then added to the mixer and mixed for a suitable amount of time. The gum can be then removed from the mixer and rolled to stick thickness on waxed paper while warm.

The compounds of Formula I may be incorporated into a system that can release a fragrance in a controlled manner. These include substrates such as air fresheners, laundry detergents, fabric softeners, deodorants, lotions, and other household items. The fragrances are generally one or more derivatives of essential oils as described herein, each present in different quantities. For example, gel articles can contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy) lower alkyl 2-alkeneoate and a polyethylenically unsaturated crosslinking agent. These materials have continuous slow release properties, i.e. they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

For any particular chemical compound disclosed herein, any general disclosure or structure presented also encompasses all isomers, such as conformational isomers, regioisomers, stereoisomers, and the like, that can arise from a particular set of substituents. The general structure also encompasses all enantiomers, diastereomers, and other optical isomers whether in enantiomeric or racemic forms, as well as mixtures of stereoisomers, as the context requires.

The following examples illustrate the aromachemcial compounds and a process for their preparation. These examples are not to be construed in any way as imposing limitations upon the scope thereof. On the contrary, it is to be clearly understood that resort can be had to various other aspects, embodiments, modifications, and equivalents thereof which, after reading the description herein, may suggest themselves to one of ordinary skill in the art.

### EXAMPLE 1

### Synthesis of Ethyl-2-cyano-3,7-dimethylocta-2,6-dienoate

Ethyl-2-cyano-3,7-dimethylocta-2,6-dienoate (Compound I) was produced by heating ethyl cyanoacetate (3.83 ml, 36.0 mmol), 6-methyl-5-hept-2-one (5 g, 39.6 mmol), acetic acid (0.41 ml, 7.2 mmol), and ammonium acetate (0.27 g, 3.6 mmol) in 100 ml of anhydrous toluene under reflux overnight in a Dean-Stark apparatus. After cooling to room temperature the organic phase was washed with water, dried over magnesium sulfate and the solvent was removed. The residue was purified by silica gel chromatography eluting with a gradient of 1-2% ethyl acetate/hexane. The desired compound, ethyl-2-cyano-3,7-dimethylocta-2,6-dienoate (compound I), was obtained as a colorless oil in 66% yield (5.3 g, 23.9 mmol).

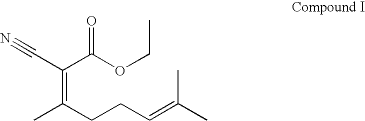

### EXAMPLE 2

### Synthesis of Ethyl-1-cyano-methyl-2-(4-methylpent-3enyl)-cycipopropanecarboxylate

Ethyl-1-cyano-methyl-2-(4-methylpent-3enyl)-cyclpopropanecarboxylate (Compound II) was produced by adding nitromethane (92.3 ml, 1.48 mol) and 1,8-Diazabicyclo[5.4.0]undec-7-ene (50.7 ml, 0.34 mol) successively to a solution of Ethyl-2-cyano-3,7-dimethylocta-2,6-dienoate (75.4 g, 0.34 mol) in 250 ml of anhydrous acetonitrile at room temperature. The reaction was stirred overnight. The acetonitrile was then removed under pressure and the residue was taken in diethyl ether. The organic layer was washed with a 1M HCl solution and dried over magnesium sulfate. The solvent was evaporated to give the desired product (Compound II) as a red/orange oil in 79% yield (72.3 g, 0.3 mol).

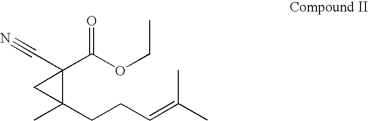

### EXAMPLE 3

### Synthesis of 1-cyano-2methyl-2-(4-methylpent-3-enyl)-cylcopropanecarboxylic acid

1-cyano-2methyl-2-(4-methylpent-3-enyl)-cylcopropanecarboxylic acid (Compound III) was produced by, first, adding a 1M sodium hydroxide solution (10 ml) to a solution of ethyl-1-cyano-methyl-2-(4-methylpent-3enyl)-cyclpopropanecarboxylate (2.3 g, 9.7 mmol) in 20 ml ethanol. The reaction mixture was stirred for 4 hours, then the ethanol was removed under vacuum. The aqueous phase was extracted with dichloromethane (DCM) (3×20 ml), then neutralized with 1M HCl and extracted with DCM (3×20 ml). The organic layer was dried over magnesium sulfate, filtered and the solvent removed under vacuum to give the desired product (Compound III) as an orange oil in 74% yield (1.51 g, 7.3 mmol).

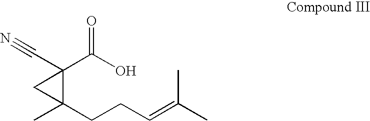

### EXAMPLE 4

### Synthesis of 2-methyl-2-(4-methyl-3-enyl)cyclopropanecarbonitrile

1-cyano-2methyl-2-(4-methylpent-3-enyl)-cylcopropanecarboxylic acid was decarboxylated by heating to 115° C. for two hours with touluene to produce 2-methyl-2-(4-methyl-3-enyl)-cyclopropanecarbonitrile (Compound IV) (˜70% yield).

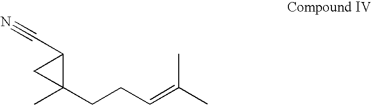

1. A process for preparing a compound of Formula I 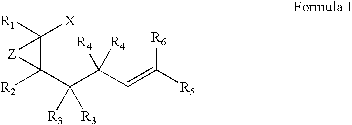
wherein R₁ is H, C(O)O—CH₂—CH₃; C(O)O—CH₃, or C(O)O—H;
R₂ is methyl;
R₃and R₄are H;
R₅ and R₆ are CH₃;
Z is C(H)₂; and
X is —CN
comprising reacting a cyanoacetate with a ketone in a condensation reaction to produce the compound of Formula I′

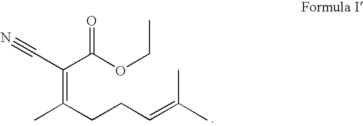

2. The process of claim 1, wherein the cyanoacetate is ethyl cyanoacetate.

3. The process of claim 1, wherein the ketone is 6-mehtyl-5-hept-2-one.

4. The process of claim 1, further comprising the step of cyclopropanating the compound of Formula I′ to produce the compound of Formula II′:

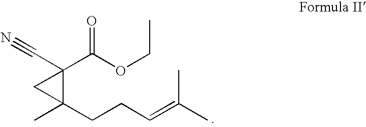

5. The process of claim 4, further comprising the step of reacting the ester of Formula II′ to produce the carboxylic acid of Formula III′.

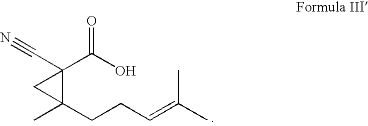

6. The process of claim 5, wherein the step of reacting the ester of Formula II′ to produce the carboxylic acid of Formula III′ is accomplished by hydrolysis.

7. The process of claim 6, wherein the step of hydrolysis is catalyzed by at least one compound chosen from an acid or base or a combination thereof.

8. The process of claim 6, further comprising the step of decarboxylating the carboxylic acid of Formula III′ to form the compound of Formula IV′.

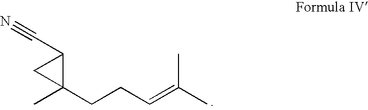

9. The process of claim 8, wherein the step of decarboxylating the carboxylic acid is accomplished by heating to a temperature of from 110° C. to 150° C.

10. The process of claim 8, wherein the step of decarboxylating the carboxylic acid is accomplished by heating to a temperature of from 110° C. to 150° C. in the presence of dimethylformamide.

11. The process of claim 8, wherein the step of decarboxylating the carboxylic acid is accomplished by reacting the compound of Formula III′ with 1,3 propane diol in the presence of NaHCO₃.

12. The process of claim 11, wherein step of decarboxylating the carboxylic acid is accomplished by heating to a temperature of from 150° C. to 210° C.

13. The process of claim 8, wherein the step of decarboxylating the carboxylic acid is accomplished by heating to a temperature of from 110° C. to 150° C. in the presence of toluene.

14. The process of claim 1, wherein the double bond attached to X is in an E configuration

15. The process of claim 1, wherein the double bond attached to X is in a Z configuration

16. A composition comprising
at least one compound of Formula I 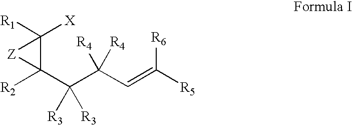
wherein R₁ is C(O)O—CH₂—CH₃; C(O)O—CH₃, or C(O)O—H;
R₂ is methyl;
R₃ and R₄ are H;
R₅ and R₆ are CH₃;
Z is C(H)₂; and
X is —CN,
wherein the at least one compound of Formula I is in admixture with at least one solvent or adjuvant or a combination thereof.

17. The composition of claim 16, wherein the composition comprises at least one article chosen from a perfume, a cologne, a beverage, a soap, a bath or shower gel, a shampoo or other hair care product, a cosmetic preparation, a body deodorant, an antiperspirant, an air freshener, a fabric detergent or softener or an all-purpose household cleaner.

18. The composition of claim 17, wherein the beverage is chosen from beer, malt liquor, lemonade or cola.
